(19) United States
(12) Patent Application Publication
(10) Pub. No.: US 2006/0005784 A1
(43) Pub. Date: Jan. 12, 2006

(54) Environmental control of confined animal rearing facilities with liquid iron compounds

(76) Inventor: Christopher B. Lind, Flanders, NJ (US)

Correspondence Address: GENERAL CHEMICAL PERFORMANCE PRODUCTS LLC., 90 EAST HALSEY ROAD, PARSIPPANY, NJ, 07054, US

(21) Appl. No.: 10/887,281

(22) Filed: Jul. 8, 2004

(51) Int. Cl. A61L 9/00; A01K 29/00

(52) U.S. Cl. 119/651; 424/76.1

(57) ABSTRACT

Liquid ferric sulfate is applied to litter, bedding or manure storage facilities in livestock and animal growing facilities for the control of ammonia, hydrogen sulfide, particulate emissions, soluble phosphorus and for animal performance improvement. Application of such materials at described rates of <50#/1000 sq. ft. to >200#/1000 sq. ft. control ammonia, odors, particulate emissions, soluble phosphorus, and improve livestock performance.

This invention relates to the enhancement of conditions prevailing in domestic animal rearing facilities and to the control of environmental effects from the manure generated at these facilities. More specifically, the present invention relates to the improvement of the atmospheric environment of animal rearing facilities that are enclosed or substantially confined. In particular, the invention deals with the problem of ammonia volatilization and odors and with soluble phosphorus runoff from manure in domestic animal rearing facilities by using a treatment comprising applying a liquid containing essentially ferric sulfate or ferric chloride or mixtures thereof.

Various building construction designs are known for housing and for sheltering livestock such as poultry, including chickens, turkeys, ducks, ostrich, swine and dairy cows in rearing facilities on farms. Conventional enclosure types, for example are such on the kind described in U.S. Pat. No. 5,890,454 comprising a flooring which includes a grate or slatted section to allow manure which accumulates to fall through the flooring opening into a manure collection pit. In such facilities the manure must be periodically cleansed, i.e. removed from the pit. Another type of facility includes an appropriately inclined floor, such as a concrete floor, which has at least one flushing trough or channel defined in the floor. Manure temporarily collects on a portion of the floor, and, in the trough, and is periodically flushed into the trough with water to wash the manure passing through the trough to an anaerobic lagoon or holding pond. A system may be installed which in this type a typical in periodically flushes and removes all of the accumulated manure at predetermined cycles, e.g., every 8 to 12 hours.

Usually these facilities are roofed and may be confined and use no absorbent bedding material permitting the manure to be relatively efficiently handled as a slurry or flowable liquid. The manure is diluted with water to the extent practical to assist in transporting and applying the manure.

In facilities of this kind, the accumulation of manure results in the production of ammonia, which accumulates and saturates the contiguous atmosphere. This ammonia is generated in relatively high quantities, and at more severe levels, is toxic to the animals as well as to humans present in substantially enclosed animal rearing spaces. Further, this ammonia reacts in the atmosphere to form particulate material (PM) of a size that is injurious to human health, PM_{2.5} and PM₁₀. The subscripts denote particle size in micrometers.

A severe related problem arising from effluent produced from the animal rearing facilities of this kind is the generation of soluble phosphorus and hydrogen sulfide, which are generated by-products that are also of substantial environmental concern.

While the prior art, e.g., U.S. Pat. No. 5,622,697 discloses the use of iron compounds including ferric and ferrous sulfates and chloride, these compounds have heretofore been suggested as being of possible utility as solids. However, the use of such iron compounds as granular solids in the animal rearing facilities suffer from severe drawbacks in that the animals are likely to ingest solids and results in illness and is often fatal. Additionally, the use of the iron compound in solid form to be distributed in practical quantities is by its nature relatively only marginally effective.

The invention deals essentially with: 1) Control of odors resulting from (NH₃) ammonia, hydrogen sulfide (H₂S), volatile fatty acids (VFA) and the like resulting from the decomposition of animal manures within the growing facility; 2) Reduction of soluble reactive phosphorus (SRP) concurrent with the odorous substance reduction. SRP is a pollutant in surface water; 3) Reductions in particulate matter emissions (PM_{2.5} and PM₁₀) concurrent with and generated by odorous substances most notably ammonia; 4) Reduction of the pH of the litter, bedding or manure to effect odor control; and 5) A mechanism to produce a sufficiently low pH and free water to inhibit proliferation of microbes and insects especially those regarded as pathogens.

To realize these objects the application of a treatment that comprises essentially liquid ferric sulfate and or chloride to the litter, to the bedding or to the manure deposited flooring, is employed. Amounts of ferric sulfate vary with type of animal waste and its physical and chemical characteristics but typical application comprise the use of about ten (10) to about thirty (30) gallons per thousand square feet of floor area surface on which the bedding or litter treatment is present or on which the livestock dwells. The treatment comprises a liquid containing from about 5% to about 15%, preferably about 8%-13% and most preferable 10%-12% ferric sulfate, as iron, in the liquid applied to the litter as flooring.

The advantage of the system of the invention over the various alternatives such as use of aluminum salts such as aluminum sulfate, aluminum chloride or aluminum nitrate, sodium bisulfate¹, clay mixed with acid (normally sulfuric), phosphoric acid, microbial preparations and enzymes, either in liquid or solid form, include the following:
1. Enzymes and microbial preparations function on the theory that the metabolic pathways of said waste decomposition can be changed to produce metabolites that are not odorous or toxic. Being biologicals they require specific growth environments and rarely demonstrate lasting effects without constant and costly reapplication. Further they do not reduce phosphorous and in fact may increase the soluble fraction through their own digestive processes thereby increasing the potentially detrimental environmental effects. Biologicals do not depress pH to control pathogens and insects rather they exhibit pH sensitivity and can die off if pH stressed. Liquid iron products are also more widely available at “commodity” pricing.
2. Phosphoric acid has been widely used in the poultry industry for acidification of the litter, floor, and bedding. Although phosphoric acid is effective, stringent phosphorous discharge and land application limits, essentially prohibit this practice. Furthermore, phosphoric acid is far more costly and is a more hazardous acid than a liquid ferric sulfate product. It is also noteworthy that liquid ferric sulfate and ferric chloride products are certified for use in drinking water assuring purity that commercial acids may not provide.
3. Acid clay mixtures are more corrosive and hazardous than liquid ferric sulfate (LFS) and some like products. Being dry products, the corrosive dusts present worker exposure issues and equipment and facility corrosion issues absent in LFS and others. Acid clay mixtures do not reduce SRP and may actually increase SRP by dissolving particulate phosphorous in manure through low pH. Acid clay mixtures do not reduce litter or bedding moisture as LFS does.
4. Sodium bisulfate (SBS) is a deliquescent salt that absorbs moisture from the air. In this manner it adds moisture to the litter—a negative aspect since reduction of free water is an essential aspect of controlling pathogens in litter. Sodium bisulfate is a dry dusty material that, like acid clay mixtures corrodes buildings and equipment and exposes workers to corrosive aerosols. SBS does not reduce SRP as LFS does. SBS contains approximately 20% (19-23%) sodium. Sodium will stay in the bedding or manure and when used as fertilizer will increase the salinity of the soil and ground water—both areas of environmental and agricultural concern. LFS contains parts per million sodium and has negligible effect on salinity. Iron is a plant nutrient and LFS treated wastes will have this nutrient as an artifact. SBS has limited utility in other applications and is produced domestically by only one producer, thus availability and pricing can be a concern.
5. Dry aluminum salts have a tendency to be dusty. While not nearly as corrosive as SBS or acid clay dusts, they can be problematic. LFS et al is applied as a liquid by custom applicators and has no dust issues. LFS is more concentrated in terms of net acidity and metal content thus fewer gallons are needed reducing potential application time. The higher iron (vs. aluminum) content results in SRP reduction at lower applied product volumes. Aluminum makes a less soluble phosphate than iron—in many cases this is desired. Some farmers prefer the slightly more soluble iron phosphate end product so they can better use the manure for its nutrient value to crop plants. There is a an apparent problem with build up of aluminum in soils that is perceived to precursor aluminum toxicity. USDA studies document the fallacy of the perception—but it still persists. Iron on the other hand is not burdened with such perceptions.
6. None of the above technologies are regarded as effective to chemically precipitate H₂S. Iron compounds on the other hand have been used for decades for precipitation of sulfides from municipal sewage, sludge and industrial wastes.

Accordingly, the need is apparent for means to alleviate the relatively toxic atmospheric in, and to manage a suitable disposal of environmentally undesirable effluent from, such facilities.

### SUMMARY OF THE INVENTION

In accordance with the invention, a novel system which employs a liquid preferably aqueous ferric sulfate or ferric chloride or mixtures thereof, preferably the ferric sulfate in concentrations at from about 5% to about 15% by weight, as iron, that deals with the environmentally undesirable conditions and effluent resulting from domestic animal rearing facilities, is provided. The system of the invention involves the addition of a suitable amount of liquid ferric sulfate to bedding material or to the water used for flushing manure collected on the animal rearing floor area or by applying the liquid ferric sulfate to a manure soiled or fresh litter or to a manure holding pit located under a slatted rearing floor. In the embodiment where liquid ferric sulfate is added to the flush water, an effective amount of the liquid ferric sulfate is dispensed into the flush water to preferably maintain the flush water at or below a pH of about 7.5, and most preferably between 4.0 and 7.0. In the embodiment where the ferric sulfate is added to a manure slurry, an amount is added depending on the accumulated quantities and maturity of the manure to preferably maintain the slurry at a concentration such that it inhibits ammonia volatilization and stabilizes, i.e. insolubilizes, the resulting soluble phosphorus.

### DETAILED DESCRIPTION OF THE INVENTION

The invention among the various advantages comprising the use of liquid ferric sulfate, or ferric chloride includes use of a readily available form of iron that is relatively safe; its activating effect is more rapid; it acts to promptly initiate the desired hydrolysis; and, in the case of litter bedding application, acidulates the litter more quickly. Liquid ferric sulfate is commonly a product produced by the digestion of an iron source (hematite, magnetite, scrap iron) with sulfuric acid. LFS can also be processed waste from steel pickling or other ferrous material manufacturing and processing. Typically the liquid ferric sulfate will have 10%-12% by weight iron and from about 0.1% to about 5.0% free acidity. It will have a specific gravity of less than about 1.3% to about 1.6% gm/cc. LFS can also be produced by dissolving various dry or solid ferric sulfate or ferrous sulfate products in water and processing to the desired final product strength and characteristics. It can also be a by-product of pigment (TiO₂) manufacturing. Liquid ferric chloride can also be utilized alone, or in combination with ferric sulfate; ferric sulfate being the preferred treatment mechanism. Ferric chloride can be produced by digestion of an iron source in hydrochloric acid or wet chlorine streams. The larger volumes are by-products from TiO₂ manufacturing or steel/iron manufacturing and processing. It will typically contain 10-12% iron and <1->3% free acid. Waste Pickle Liquor (WPL) is a ferric/ferrous chloride/sulfite by product of iron and steel manufacturing and processing. It is variable in chemical assay and strength tending to low (<5%) iron content and high (>5%) acidity. It is often used as a starting material for other products herein such as ferrous chloride or ferrous sulfate.

Liquid ferric sulfate normally may be processed from a ferric sulfate manufacturing plant, or from industrial waste acid streams or through dissolving scrap iron in sulfuric acid by diluting or concentrating the desired strength. Substituting hydrochloric acid for the sulfuric acid in the previous manner produces ferric chloride and ferrous chloride materials also suitable. Ferric sulfate is also available as dry materials from iron and pigment manufacturers and when utilized can be solubilized in water to the appropriate concentration for use.

In essence the invention provides an environment enhancing system comprising applying a compound selected from liquid ferric sulfate, liquid ferric chloride and mixtures thereof in amounts effective to suitably treat the type of animal waste and its physical and chemical characteristics and within limits of from about ten (10) to thirty (30) gallons containing about 5% to about 15% by weight of iron per thousand square feet of floor area of bedding or litter surface in which the livestock such as, poultry, swine or cattle are housed.

The invention will be further described by reference to the following examples. However, the invention is not meant to be limited to the details described therein. In the examples, parts and percentages are by weight unless otherwise noted.

### EXAMPLE 1

Use of liquid ferric sulfate as a litter amendment for broiler chicken manure.

One hundred grams of one-year old used broiler litter was added to 900 ml deionized water and mixed 30 minutes using a six place Phipps and Bird model 7790-400 jar stirrer. The litter sample was obtained Mar. 08, 2004 from a commercial operation in Springdale, Ark. growing Cobb-Vantress birds six weeks. Five flocks of 20,000-30,000 birds were raised on this rice hull litter which was decaked (manure mats removed partially) and top-dressed (replenished) with fresh rice hulls after every flock. No litter amendments were used.

To each of six samples aluminum sulfate was added as a known effect at doses per liter of litter and water of 0 (control) 1 g, 2.5 g, 5 g, 10 g and 20 g. The test data with LFS at 12.1% Fe were based on 0.5 g/1.0 g, 2.5 g, 5 g, 10 g and 20 g. The pH was determined on samples settled for one hour using an Orion Ionanalyzer Model EA920. Target pH is <6 for ammonia control. SRP was determined on a Hach Model 780 Spectrophotometer using the Phosver®3 method. SRP was determined on diluted samples of litter. Deionized and distilled reagent blanks were also run.

| Dose | | Alum | | 12% LFS |
|---|---|---|---|---|
| g/l | pH | SRP | PH | SRP |
| 0 | 8.06 | 110 | 8.06 | 110 |
| 0.5 | — | — | 7.93 | 60 |
| 1.0 | 7.70 | 76 | 7.70 | 46 |
| 2.5 | 7.41 | 35 | 6.81 | 45 |
| 5.0 | 6.88 | 16 | 5.81 | 34 |
| 10.0 | 5.72 | 16 | 4.15 | 10 |
| 20.0 | 4.55 | 16 | 3.22 | 27 |

At equivalent dosages in grams of product per liter of sample LFS produced a low pH. Alum still produced generally lower SRP values—but LFS produced the lowest SRP. LFS produced the desired results.

### EXAMPLE 2

Based on experimental data a commercial broiler house(s) will be treated with LFS at 0, 7.5 gallons/1000 square fee and 15 gallons/1000 square feet. This is equivalent to 100# dry alum/1000 square feet and 200# dry alum/1000 square feet or 25 and 50 gallons of 7% liquid acid alum respectively. Analytical data include ammonia, pH of litter, SRP of litter, bird growth statistics including feed conversion, mortality and condemnation rate, insect infestation and litter microbiology.

### EXAMPLE 3

After total clean out of commercial broiler houses the dirt floors (pad) will be treated with 0, 7.5 gallons, 15 gallons per 1000 square feet or liquid ferric sulfate containing 10% to 12% as Fe. Analytical data will include soil pH at 0, 2″ and 6″, soil SRP, soil organic content and microbiology.

The use of liquid alum for ammonia, particulates, and SRP control is a significantly increasing application for litter amendment product. A 7% acid product being the leader. By virtue of its higher acidity and metal content LFS et al can be shipped greater distances to poultry or swine areas not easily served by liquid alum plants. SRP regulations on agricultural wastes go into effect in 2006 with agricultural air quality enforceable regulations 2-4 years later. The advantages of the LFS system should expand into the market holes not reachable by alum or competitive products either by freight or functionality.

### EXAMPLE 4

Use of liquid ferric sulfate is projected in a commercial swine raising house deep manure pit. These deep pits are beneath the swine and the decomposition of manure, urine and spilled feed generates considerable odors. After analysis of manure pit contents and house atmosphere, LFS is sprayed directly under the slats the swine stand on. This spray will have the effect of scrubbing ammonia and hydrogen sulfide from the air as well as treating the contents of the manure pits. Dosage rates will be determined based on stoichiometric rates of iron for hydrogen sulfite control and multiples thereof. That is stoichiometric Fe×1; 1.5, 2, 2.5 and the like.

Additional determinations will be focused on the required LFS to depress and maintain pH of manure to <6.0 for ammonia control.

Soluble reactive P will be determined on treated and untreated manure samples.

Although the invention has been described in terms of particular embodiments, blends of one or more of the various additives described herein can be used, and substitutes therefor, as will be known to those skilled in the art. Thus the invention is not meant to be limited to the details described herein, but only by the scope of the appended claims.

What is claimed:

1. A method for treating a hostile animal rearing environment contaminated with odors from (NH₃) ammonia, hydrogen sulfide (H₂S), volatile fatty acids (VFA) and the like resulting from the decomposition of animal manures within a substantially confined growing facility to reduce ammonia levels and soluble reactive phosphorus (SRP) concurrent with the odorous substance reduction and to reduce the pH of the litter, bedding or manure to effect the odor and produce a sufficiently low pH and free water to inhibit proliferation of microbes and insects, the improvement comprising applying a compound selected from liquid ferric sulfate, liquid ferric chloride and mixtures thereof in amounts effective to suitably treat the type of animal waste and its physical and chemical characteristics and within limits of from about ten (10) to thirty (30) gallons containing about 5% to about 15% by weight of iron per thousand square feet of floor area of bedding or litter surface.

2. The method of claim 1 wherein the concentration of the ferric sulfate, ferric chloride or mixtures is from about 10% to about 12% as iron and <1% to 20% by weight in liquid to solid manures.

3. The method of claim 2 as applied to poultry litter.

4. The method of claim 3 as applied to the litter for poultry selected from chickens, turkeys and ducks.

5. The method of claim 2 as applied to swine.

6. The method of claim 2 as applied to dairy cows.

7. The method of claim 1 wherein the selected ferric sulfate, ferric chloride and mixtures thereof is applied to flush water and is present in sufficient concentrations to maintain the flush water at a pH of below about 7.5.

8. The method of claim 7 wherein the ferric sulfate is selected and applied in concentrations sufficient to maintain the pH between about 4.0 and 7.0.

9. The method of claim 1 wherein the compound selected is ferric sulfate that comprises about 10% to about 12% by weight iron and a specific gravity less than about 1.3 to about 1.5.

10. The method of claim 9 wherein the applied liquid sulfate has a pH of between about 4.0 and 7.0.
